(19) United States
(12) Patent Application Publication
(10) Pub. No.: US 2009/0239918 A1
(43) Pub. Date: Sep. 24, 2009

(54) SELECTIVE SUBTYPE ALPHA 2 ADRENERGIC AGENTS AND METHODS FOR USE THEREOF

(75) Inventors: Janet A. Takeuchi, Anaheim, CA (US); Ling Li, Irvine, CA (US); Todd M. Heidelbaugh, Fountain Valley, CA (US); Ken Chow, Newport Coast, CA (US); Karen M. Kedzie, Rancho Santa Margarita, CA (US); Daniel W. Gil, Corona Del Mar, CA (US); Wenkui K. Fang, Irvine, CA (US)

Correspondence Address: ALLERGAN, INC., 2525 DUPONT DRIVE, T2-7H, IRVINE, CA, 92612-1599, US

(73) Assignee: ALLERGAN, INC., Irvine, CA (US)

(21) Appl. No.: 12/408,823

(22) Filed: Mar. 23, 2009

Related U.S. Application Data
(60) Provisional application No. 61/038,928, filed on Mar. 24, 2008.

(51) Int. Cl. A61K 31/426; A61K 31/4164; A61K 31/421

(52) U.S. Cl. 514/370; 514/385; 514/401; 514/377

(57) ABSTRACT

The invention provides methods for treating pain in mammals. In particular, the invention provides well-defined aminoimidazolines, aminothiazolines, and aminooxazolines and pharmaceutical compositions thereof to treat pain.

### RELATED APPLICATION

This application claims the benefit of U.S. Provisional Application Ser. No. 61/038,928, filed Mar. 24, 2008, the disclosure of which is hereby incorporated in its entirety herein by reference.

### FIELD OF THE INVENTION

The present invention relates generally to methods for treating various types of pain in mammals. The invention relates specifically to the use of certain aminoimidazoline, aminothiazoline, and aminooxazoline compounds and pharmaceutical compositions thereof to treat pain.

### BACKGROUND OF THE INVENTION

Human adrenergic receptors are integral membrane proteins that have been classified into two broad classes, the alpha and the beta adrenergic receptors. Both types mediate the action of the peripheral sympathetic nervous system upon binding of catecholamines, norepinephrine and epinephrine.

Norepinephrine is produced by adrenergic nerve endings, while epinephrine is produced by the adrenal medulla. The binding affinity of adrenergic receptors for these compounds forms one basis of the classification: alpha receptors tend to bind norepinephrine more strongly than epinephrine and much more strongly than the synthetic compound isoproterenol. The preferred binding affinity of these hormones is reversed for the beta receptors. In many tissues, the functional responses, such as smooth muscle contraction, induced by alpha receptor activation are opposed to responses induced by beta receptor binding.

Subsequently, the functional distinction between alpha and beta receptors was further highlighted and refined by the pharmacological characterization of these receptors from various animal and tissue sources. As a result, alpha and beta adrenergic receptors were further subdivided into alpha 1, alpha 2, beta 1, and beta 2 subtypes. Functional differences between alpha 1 and alpha 2 receptors have been recognized, and compounds that exhibit selective binding between these two subtypes have been developed. Thus, in published international patent application WO 92/0073, the selective ability of the R(+) enantiomer of terazosin to selectively bind to adrenergic receptors of the alpha 1 subtype was reported. The alpha 1/alpha 2 selectivity of this compound was disclosed as being significant because agonist stimulation of the alpha 2 receptors was said to inhibit secretion of epinephrine and norepinephrine, while antagonism of the alpha 2 receptor was said to increase secretion of these hormones. Thus, the use of non-selective alpha-adrenergic blockers, such as phenoxybenzamine and phentolamine, was said to be limited by their alpha 2 adrenergic receptor mediated induction of increased plasma catecholamine concentration and the attendant physiological sequelae (increased heart rate and smooth muscle contraction).

For a further general background on the alpha-adrenergic receptors, the reader's attention is directed to Robert R. Ruffolo, Jr., alpha-Adrenoreceptors: Molecular Biology, Biochemistry and Pharmacology, (Progress in Basic and Clinical Pharmacology series, Karger, 1991), wherein the basis of alpha 1/alpha 2 subclassification, the molecular biology, signal transduction, agonist structure-activity relationships, receptor functions, and therapeutic applications for compounds exhibiting alpha-adrenergic receptor affinity is explored.

The cloning, sequencing and expression of alpha receptor subtypes from animal tissues has led to the subclassification of the alpha 1 adrenoreceptors into alpha 1A, alpha 1B and alpha 1D. Similarly, the alpha 2 adrenoreceptors have also been classified alpha 2A, alpha 2B, and alpha 2C receptors. Each alpha 2 receptor subtype appears to exhibit its own pharmacological and tissue specificities. Compounds having a degree of specificity for one or more of these subtypes may be more specific therapeutic agents for a given indication than an alpha 2 receptor pan-agonist (such as the drug clonidine) or a pan-antagonist.

Among other indications, such as the treatment of glaucoma, hypertension, sexual dysfunction, and depression, certain compounds having alpha 2 adrenergic receptor agonist activity are known analgesics. However, many compounds having such activity do not provide the activity and specificity desirable when treating disorders modulated by alpha 2 adrenoreceptors. For example, many compounds found to be effective agents in the treatment of pain are frequently found to have undesirable side effects, such as causing hypotension and sedation at systemically effective doses. There is a need for new drugs that provide relief from pain without causing these undesirable side effects. Additionally, there is a need for agents which display activity against pain, particularly chronic pain, such as chronic neuropathic and visceral pain.

### SUMMARY OF THE INVENTION

The invention provides methods for treating pain in mammals. In particular, the invention provides well-defined aminoimidazolines, aminothiazolines, and aminooxazolines and pharmaceutical compositions thereof to treat pain.

In one embodiment of the invention, there are provided methods for treating pain. Such methods can be performed, for example, by administering to a mammal in need thereof a pharmaceutical composition containing a therapeutically effective amount of at least one compound having the structure:

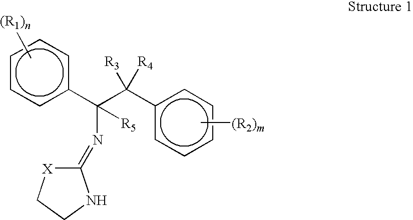

wherein:
   X is O, S, or NH;
   n and m are each independently 1 to 5;
   each R₁ and R₂ is independently H, alkyl, cycloalkyl, aryl, alkenyl, alkynyl, halide, hydroxy, alkoxy, trifluoromethyl, —N(R₆)₂, —CN, —CO₂R₆, or —CH₂OH; and
   R₃, R₄, R₅, and R₆ are each independently H or lower alkyl;
      or any combination thereof, or pharmaceutically acceptable salts, hydrates, solvates, crystal forms, isomers, tautomers, enantiomers, and diastereomers thereof.

### DETAILED DESCRIPTION OF THE INVENTION

It is to be understood that both the foregoing general description and the following detailed description are exemplary and explanatory only and are not restrictive of the invention claimed. As used herein, the use of the singular includes the plural unless specifically stated otherwise. As used herein, “or” means “and/or” unless stated otherwise. Furthermore, use of the term “including” as well as other forms, such as “includes,” and “included,” is not limiting. The section headings used herein are for organizational purposes only and are not to be construed as limiting the subject matter described.

Unless specific definitions are provided, the nomenclatures utilized in connection with, and the laboratory procedures and techniques of analytical chemistry, synthetic organic and inorganic chemistry described herein are those known in the art. Standard chemical symbols are used interchangeably with the full names represented by such symbols. Thus, for example, the terms “hydrogen” and “H” are understood to have identical meaning. Standard techniques may be used for chemical syntheses, chemical analyses, and formulation.

As used herein, “alkyl” refers to straight or branched chain hydrocarbyl groups having from 1 up to about 100 carbon atoms. Whenever it appears herein, a numerical range, such as “1 to 100” or “C₁-C₁₀₀”, refers to each integer in the given range; e.g., “C₁-C₁₀₀ alkyl” means that an alkyl group may comprise only 1 carbon atom, 2 carbon atoms, 3 carbon atoms, etc., up to and including 100 carbon atoms, although the term “alkyl” also includes instances where no numerical range of carbon atoms is designated. “Substituted alkyl” refers to alkyl moieties bearing substituents including alkyl, alkenyl, alkynyl, hydroxy, oxo, alkoxy, mercapto, cycloalkyl, substituted cycloalkyl, heterocyclic, substituted heterocyclic, aryl, substituted aryl, heteroaryl, substituted heteroaryl, aryloxy, substituted aryloxy, halogen, haloalkyl, cyano, nitro, nitrone, amino, lower alkylamino, lower alkyldiamino, amido, azido, —C(O)H, —C(O)R₇, —CH₂OR₇, —C(O)—, —C(O)—, —S—, —S(O)₂, —OC(O)—O—, wherein R₇ is H or lower alkyl, acyl, oxyacyl, carboxyl, carbamate, sulfonyl, sulfonamide, sulfuryl, and the like. As used herein, “lower alkyl” refers to alkyl moieties having from 1 to about 6 carbon atoms.

As used herein, “alkenyl” refers to straight or branched chain hydrocarbyl groups having at least one carbon-carbon double bond, and having in the range of about 2 up to about 100 carbon atoms, and “substituted alkenyl” refers to alkenyl groups further bearing one or more substituents as set forth above. As used herein, “lower alkenyl” refers to alkenyl moieties having from 2 to about 6 carbon atoms.

As used herein, “alkynyl” refers to straight or branched chain hydrocarbyl groups having at least one carbon-carbon triple bond, and having in the range of about 2 up to about 100 carbon atoms, and “substituted alkynyl” refers to alkynyl groups further bearing one or more substituents as set forth above. As used herein, “lower alkynyl” refers to alkynyl moieties having from 2 to about 6 carbon atoms.

As used herein, “cycloalkyl” refers to cyclic (i.e., ring-containing) alkyl moieties typically containing in the range of about 3 up to about 8 carbon atoms, and “substituted cycloalkyl” refers to cycloalkyl groups further bearing one or more substituents as set forth above.

As used herein, “aryl” refers to aromatic groups having in the range of 6 up to 14 carbon atoms and “substituted aryl” refers to aryl groups further bearing one or more substituents as set forth above.

As used herein, “heteroaryl” refers to aromatic moieties containing one or more heteroatoms (e.g., N, O, S, or the like) as part of the ring structure and having in the range of 5 up to 14 total atoms in the ring structure (i.e., carbon atoms and heteroatoms). “Substituted heterocyclic” refers to heterocyclic groups further bearing one or more substituents as set forth above.

As used herein, “heterocyclic” refers to non-aromatic cyclic (i.e., ring-containing) groups containing one or more heteroatoms (e.g., N, O, S, or the like) as part of the ring structure, and having in the range of 3 up to 14 carbon atoms and “substituted heterocyclic” refers to heterocyclic groups further bearing one or more substituents as set forth above.

As used herein, “halogen” or “halide” refers to fluoride, chloride, bromide or iodide.

It will be readily apparent to those skilled in the art that some of the compounds of the invention may contain one or more asymmetric centers, such that the compounds may exist in enantiomeric as well as in diastereomeric forms. Unless it is specifically noted otherwise, the scope of the present invention includes all enantiomers, diastereomers and racemic mixtures. Some of the compounds of the invention may form salts with pharmaceutically acceptable acids or bases, and such pharmaceutically acceptable salts of the compounds described herein are also within the scope of the invention.

In addition, the compounds represented by Structure 1 can undergo tautomeric transformations and can be depicted by the tautomeric structures shown below. Referring to Structure 1, when X is N, the following tautomers are possible:

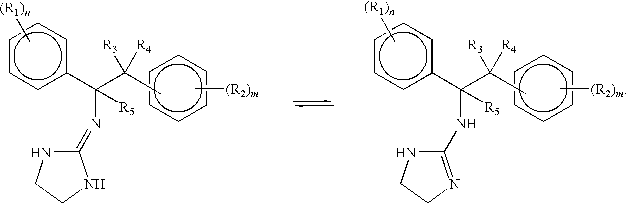

When X is S, the following tautomers are possible:

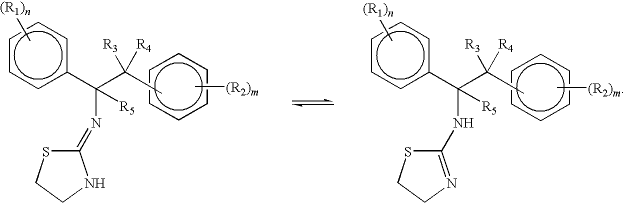

When X is O, the following tautomers are possible:

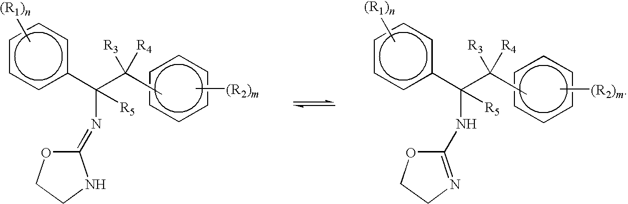

All tautomers of Structure 1 are within the scope of the invention.

A “pharmaceutically acceptable salt” is any salt that retains the activity of the parent compound and does not impart any additional deleterious or untoward effects on the subject to which it is administered and in the context in which it is administered compared to the parent compound. A pharmaceutically acceptable salt also refers to any salt which may form in vivo as a result of administration of an acid, another salt, or a prodrug which is converted into an acid or salt.

Pharmaceutically acceptable salts of acidic functional groups may be derived from organic or inorganic bases. The salt may comprise a mono or polyvalent ion. Of particular interest are the inorganic ions, lithium, sodium, potassium, calcium, and magnesium. Organic salts may be made with amines, particularly ammonium salts such as mono-, di- and trialkyl amines or ethanol amines. Salts may also be formed with caffeine, tromethamine and similar molecules. Hydrochloric acid or some other pharmaceutically acceptable acid may form a salt with a compound that includes a basic group, such as an amine or a pyridine ring.

A “prodrug” is a compound which is converted to a therapeutically active compound after administration, and the term should be interpreted as broadly herein as is generally understood in the art. While not intending to limit the scope of the invention, conversion may occur by hydrolysis of an ester group or some other biologically labile group. Generally, but not necessarily, a prodrug is inactive or less active than the therapeutically active compound to which it is converted.

The invention provides methods for treating pain. Such methods can be performed, for example, by administering to a mammal in need thereof a pharmaceutical composition containing a therapeutically effective amount of at least one compound having the structure:

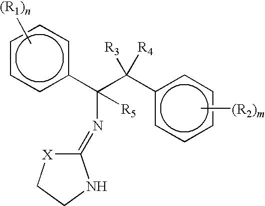

wherein:
   X is O, S, or NH;
   n and m are each independently 1 to 5;
   each R₁ and R₂ is independently H, alkyl, cycloalkyl, aryl, alkenyl, alkynyl, halide, hydroxy, alkoxy, trifluoromethyl, —N(R₆)₂, —CN, —CO₂R₆, or —CH₂OH; and
   R₃, R₄, R₅, and R₆ are each independently H or lower alkyl;
      or any combination thereof, or pharmaceutically acceptable salts, hydrates, solvates, crystal forms, isomers, tautomers, enantiomers, and diastereomers thereof.

In some embodiments, the compounds used in the methods of the invention include compounds wherein each R₁ and R₂ is independently H, lower alkyl, fluoro, chloro, bromo, trifluoromethyl, hydroxy, or methoxy. In certain embodiments, the invention methods employ compounds wherein each R₁ and R₂ is independently H, lower alkyl, or chloro.

In some embodiments invention methods employ compounds wherein X is S. Compounds according to this embodiment of the invention include, but are not limited to, compounds having the structures set forth below:

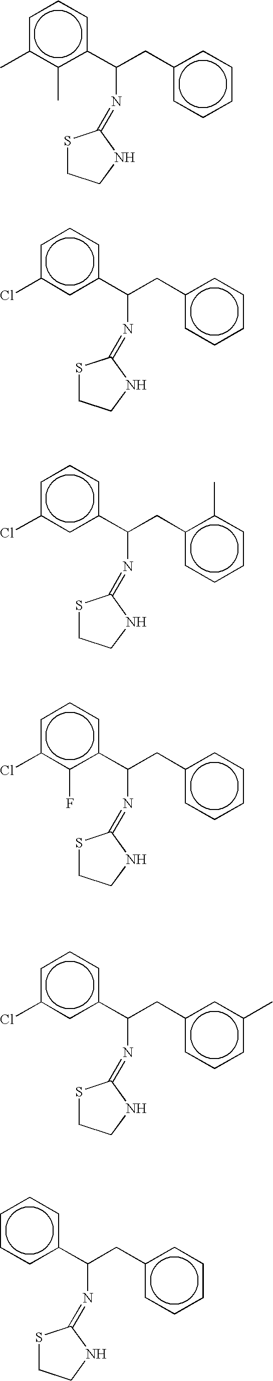

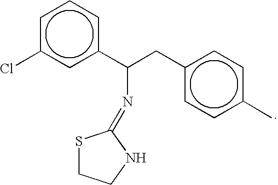

In some embodiments invention methods employ compounds wherein X is NH. Compounds according to this embodiment of the invention include, but are not limited to, compounds having the structures set forth below:

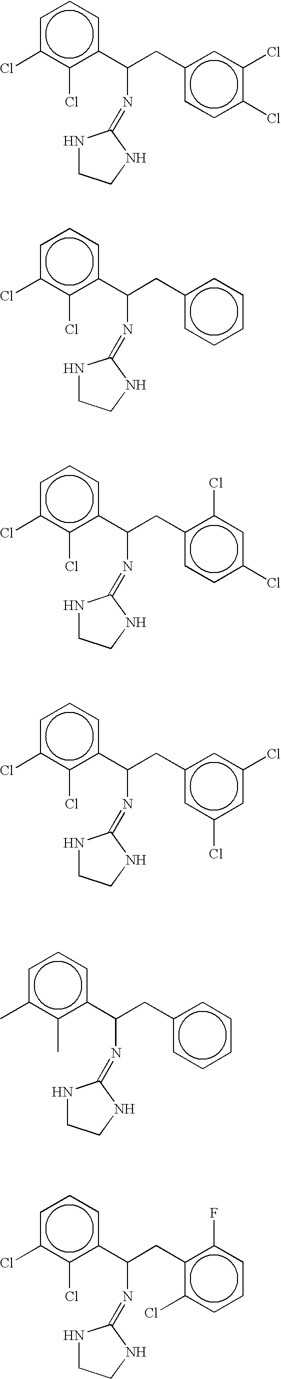

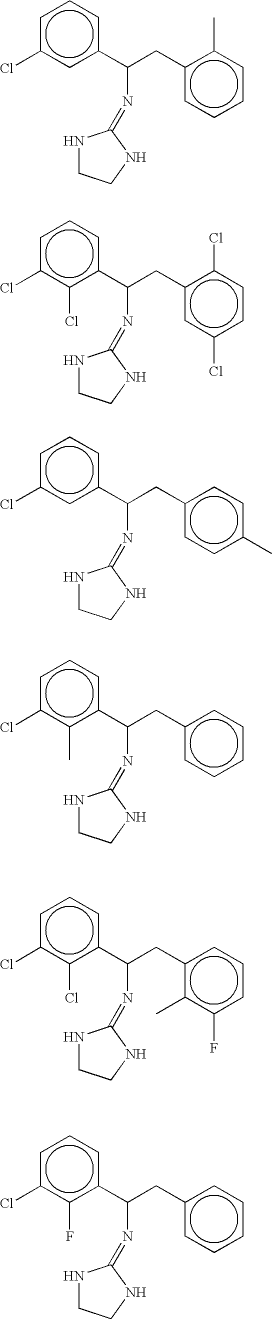

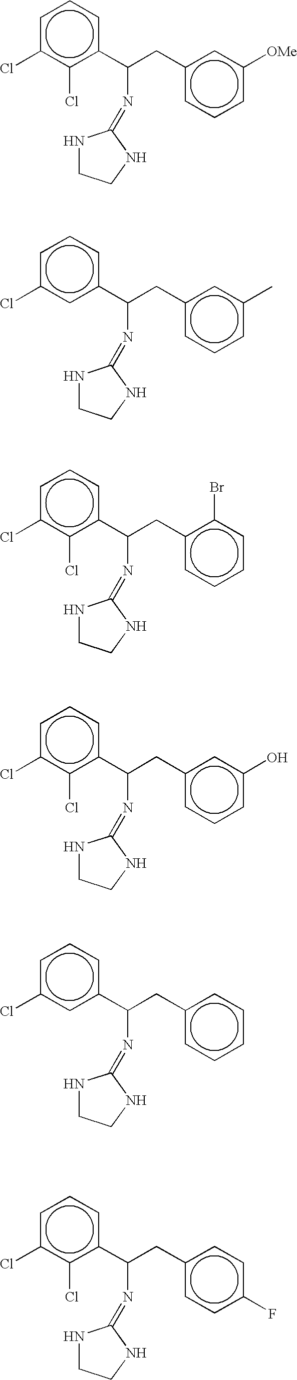

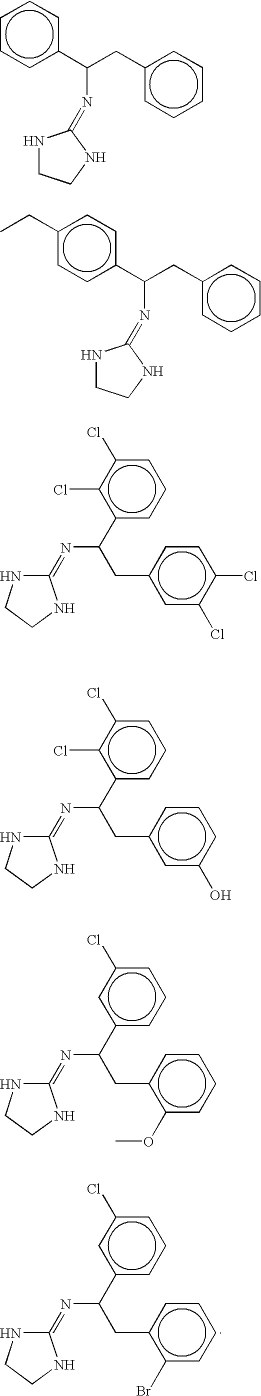

In some embodiments invention methods employ compounds wherein X is O. Compounds according to this embodiment of the invention include, but are not limited to, compounds having the structures set forth below:

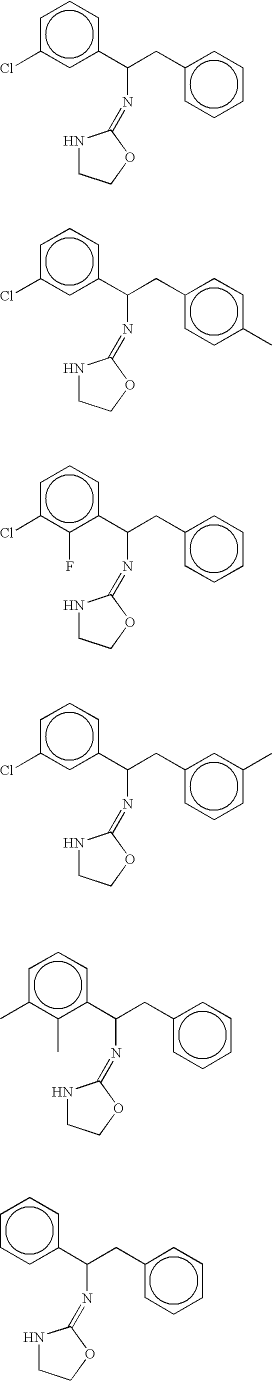

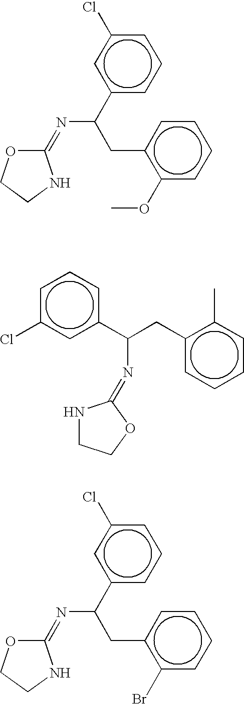

The compounds set forth herein are typically prepared by reacting appropriately substituted amines with isocyanates, isothiocyanates, or imidazoline sulfonic acids. Scheme A outlined below describes an exemplary synthesis of a precursor amine used in preparing invention compounds. Experimental details are set forth in the Examples, vide infra.

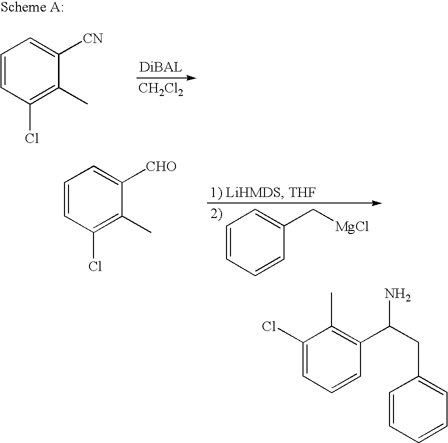

Coupling of the amines with either isocyanate, isothiocyanate, or imidazole sulfonic acids can be achieved as set forth below in Schemes 1-3.

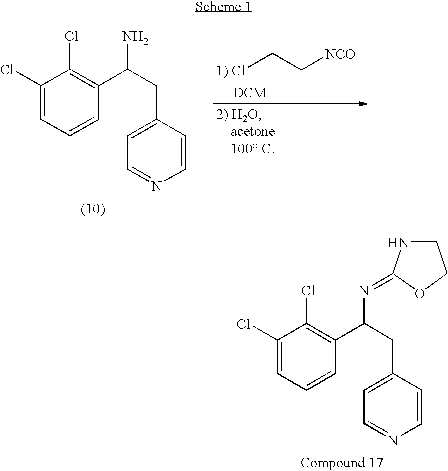

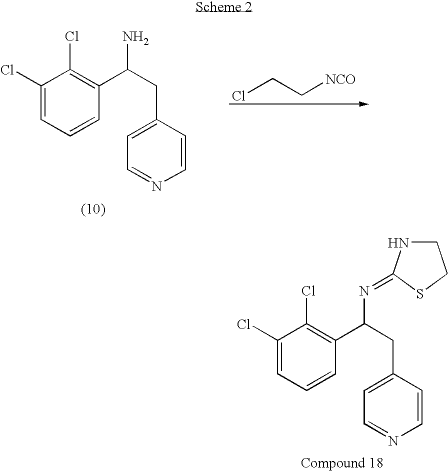

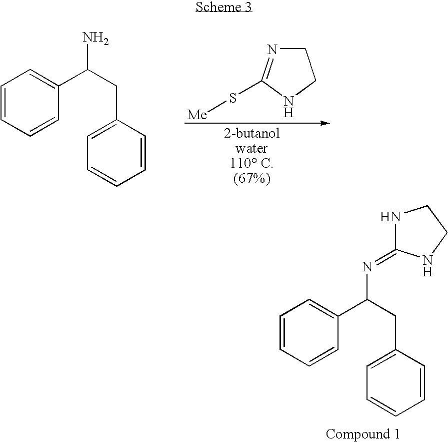

The alpha 2 adrenergic activity of the compounds employed by invention methods is demonstrated in an assay titled Receptor Selection and Amplification technology (RSAT) assay, which is described in the publication by Messier et. al., 1995, Pharmacol. Toxicol. 76, pp. 308-311 (incorporated herein by reference) and is also described below.

The RSAT assay measures a receptor-mediated loss of contact inhibition that results in selective proliferation of receptor-containing cells in a mixed population of confluent cells. The increase in cell number is assessed with an appropriate transfected marker gene such as β-galactosidase, the activity of which can be easily measured in a 96-well format. Receptors that activate the G protein, Gq, elicit this response. Alpha 2 receptors, which normally couple to Gi, activate the RSAT response when coexpressed with a hybrid Gq protein that has a Gi receptor recognition domain, called Gq/i5.

NIH-3T3 cells are plated at a density of 2×10⁶ cells in 15 cm dishes and maintained in Dulbecco's modified Eagle's medium supplemented with 10% calf serum. One day later, cells are cotransfected by calcium phosphate precipitation with mammalian expression plasmids encoding p-SV-β-galactosidase (5-10 μg), receptor (1-2 μg) and G protein (1-2 μg). 40 μg salmon sperm DNA may also be included in the transfection mixture. Fresh media is added on the following day and 1-2 days later, cells are harvested and frozen in 50 assay aliquots. Cells are thawed and 100 μl added to 100 μL aliquots of various concentrations of drugs in triplicate in 96-well dishes. Incubations continue 72-96 hr at 37° C. After washing with phosphate-buffered saline, β-galactosidase enzyme activity is determined by adding 200 μL of the chromogenic substrate (consisting of 3.5 mM o-nitrophenyl-β-D-galactopyranoside and 0.5% nonidet P-40 in phosphate buffered saline), incubating overnight at 30° C. and measuring optical density at 420 nm. The absorbance is a measure of enzyme activity, which depends on cell number and reflects a receptor-mediated cell proliferation. The efficacy or intrinsic activity is calculated as a ratio of the maximal effect of the drug to the maximal effect of a standard full agonist for each receptor subtype. Brimonidine, the chemical structure of which is shown below, is used as the standard agonist for the alpha 2B and alpha 2C receptors.

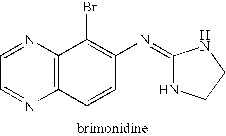

The results of the RSAT assay with several exemplary compounds employed by invention methods are disclosed in Table 1 below, together with the chemical structures of these exemplary compounds.

| Biological Data: Intrinsic Activity | | | |
|---|---|---|---|
| RSAT | | | |
| EC50 (nM) | | | |
| (rel eff) | Alpha | Alpha | Alpha |
| na = not active | 2A | 2B | 2C |
| 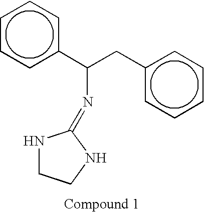 | na | 6.38 (0.77) | na |
| 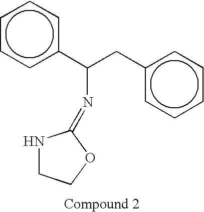 | na | 3.69 (0.95) | 3.36 (0.45) |
| 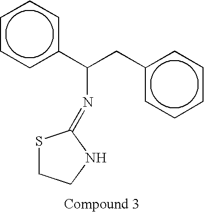 | na | 5.43 (0.90) | 9.63 (0.42) |
| 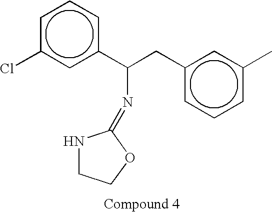 | na | 2.79 (0.92) | 8.56 (0.63) |
| 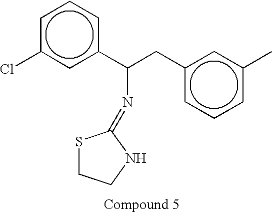 | na | 2.96 (0.82) | 4.33 (0.44) |
| 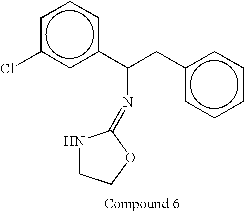 | na | 7.14 (0.83) | 4.39 (0.37) |
| 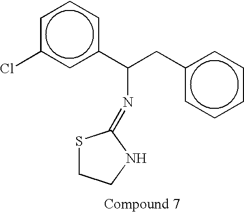 | na | 6.49 (0.89) | 4.71 (0.33) |
| 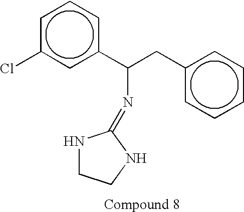 | na | 5.91 (0.76) | na |
| 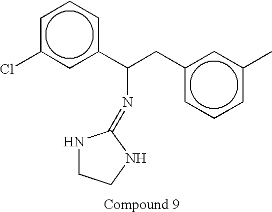 | na | 1.52 (0.81) | 3.70 (0.38) |
| 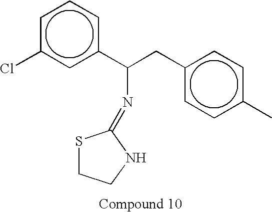 | na | 7.32 (0.76) | 11.80 (0.45) |
| 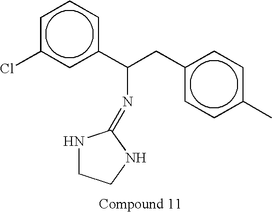 | na | 5.61 (0.69) | na |
| 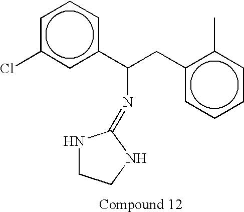 | na | 1.92 (0.87) | na |
| 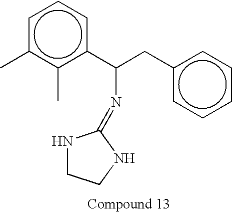 | na | 2.54 (0.83) | na |
| 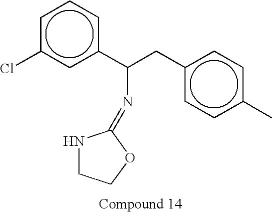 | na | 5.11 (0.74) | 6.78 (0.46) |
| 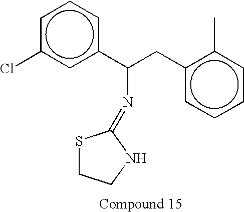 | na | 3.29 (0.89) | 8.34 (0.46) |
| 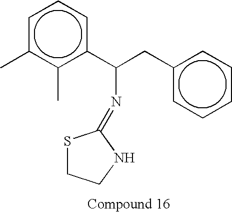 | na | 3.69 (0.89) | 9.48 (0.45) |
| 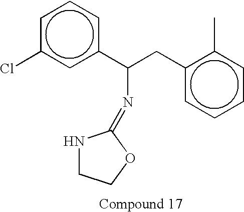 | na | 1.52 (0.87) | 1.46 (0.39) |
| 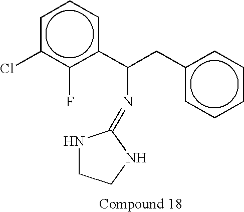 | na | 3.57 (0.85) | 1.97 (0.39) |
| 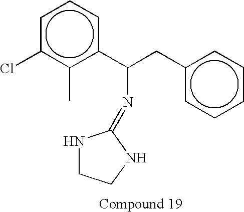 | na | 7.64 (0.77) | na |
| 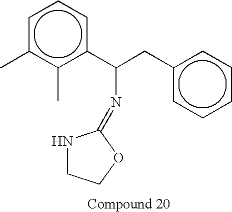 | na | 3.48 (0.89) | 13.35 (0.39) |
| 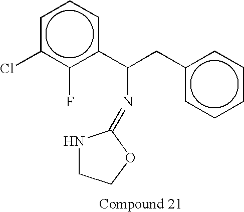 | na | 2.79 (0.93) | 2.57 (0.32) |
| 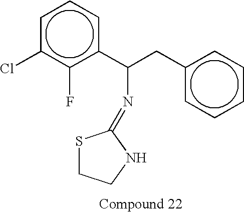 | na | 4.54 (0.89) | 6.48 (0.38) |
| 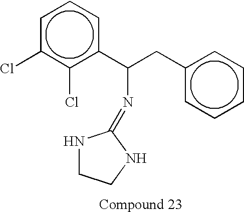 | na | 3.09 (0.89) | na |
| 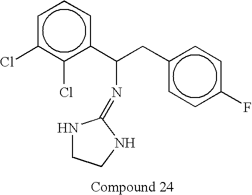 | na | 3.73 (0.89) | na |
| 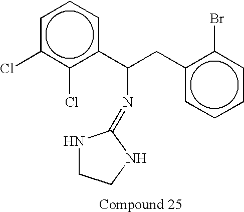 | na | 3.24 (0.89) | na |
| 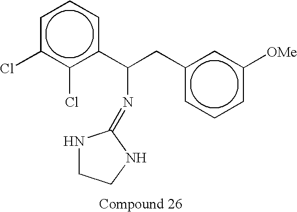 | na | 1.91 (1.00) | na |
| 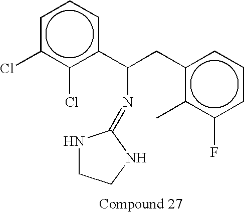 | na | 8.04 (0.95) | 3.54 (0.31) |
| 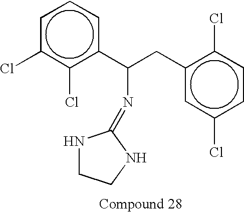 | na | 1.02 (0.98) | 3.92 (0.39) |
| 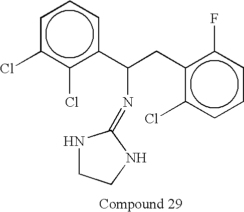 | na | 12.46 (0.89) | na |
| 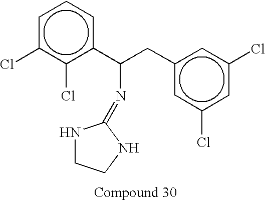 | na | 0.90 (1.20) | 5.74 (0.42) |
| 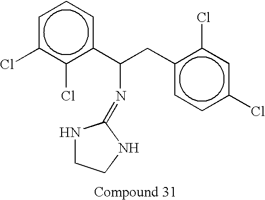 | na | 2.79 (1.17) | na |
| 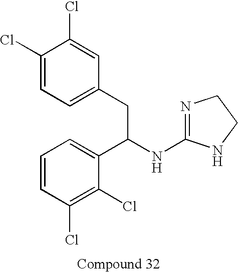 | na | 10.78 (0.96) | na |
| 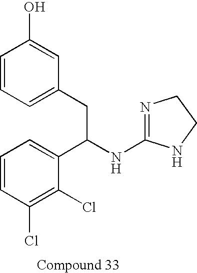 | na | 1.55 (1.05) | na |
| 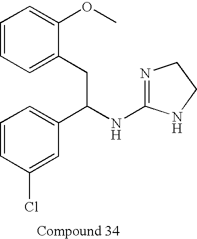 | na | 0.61 (0.89) | na |
| 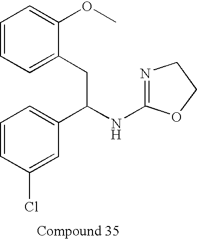 | na | 0.63 (0.91) | na |
| na | na | 0.66 (0.84) | |
| 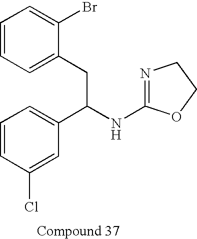 | na | 0.86 (0.92) | na |

The methods of the invention are useful in treating pain, including acute pain and chronic pain. By “acute pain” is meant immediate, usually high threshold pain brought about by injury such as a cut, crush, burn, or by chemical stimulation such as that experienced upon exposure to capsaicin, the active ingredient in chili peppers. By “chronic pain” is meant pain other than acute pain, such as, without limitation, neuropathic pain, visceral pain (including that brought about by Crohn's disease, irritable bowel syndrome (IBS), functional dyspepsia, and the like), and referred pain.

It is known that chronic pain (such as pain from cancer, arthritis, and many neuropathic injuries) and acute pain (such as that pain produced by an immediate mechanical stimulus, such as tissue section, pinch, prick, or crush) are distinct neurological phenomena mediated to a large degree either by different nerve fibers and neuroreceptors or by a rearrangement or alteration of the function of these nerves upon chronic stimulation. Sensation of acute pain is transmitted quite quickly, primarily by afferent nerve fibers termed C fibers, which normally have a high threshold for mechanical, thermal, and chemical stimulation. While the mechanisms of chronic pain are not completely understood, acute tissue injury can give rise within minutes or hours after the initial stimulation to secondary symptoms, including a regional reduction in the magnitude of the stimulus necessary to elicit a pain response. This phenomenon, which typically occurs in a region emanating from (but larger than) the site of the original stimulus, is termed hyperalgesia. The secondary response can give rise to profoundly enhanced sensitivity to mechanical or thermal stimulus.

The A afferent fibers (Aβ and Aδ fibers) can be stimulated at a lower threshold than C fibers, and appear to be involved in the sensation of chronic pain. For example, under normal conditions, low threshold stimulation of these fibers (such as a light brush or tickling) is not painful. However, under certain conditions such as those following nerve injury or in the herpes virus-mediated condition known as shingles the application of even such a light touch or the brush of clothing can be very painful. This condition is termed allodynia and appears to be mediated at least in part by Aβ afferent nerves. C fibers may also be involved in the sensation of chronic pain, but if so it appears clear that persistent firing of the neurons over time brings about some sort of change which now results in the sensation of chronic pain.

The methods of the invention employ compounds and/or pharmaceutically acceptable compositions administered at pharmaceutically effective dosages. Such dosages are normally the minimum dose necessary to achieve the desired therapeutic effect; for example, in the treatment of chronic pain, this amount would be roughly that necessary to reduce the discomfort caused by the pain to tolerable levels. Generally, such doses will be in the range 1-1000 mg/day; more preferably in the range 10 to 500 mg/day. However, the actual amount of the compound and/or composition to be administered in any given case will be determined by a physician taking into account the relevant circumstances, such as the severity of the pain, the age and weight of the patient, the patient's general physical condition, the cause of the pain, and the route of administration.

The methods of the invention are useful in the treatment of pain in a mammal, particularly a human being. In certain cases, the patient will be given a compound and/or pharmaceutical composition orally in any acceptable form, such as a tablet, liquid, capsule, powder and the like. However, other routes may be desirable or necessary, particularly if the patient suffers from nausea. Such other routes may include, without exception, transdermal, parenteral, subcutaneous, intranasal, intrathecal, intramuscular, intravenous, and intrarectal modes of delivery. Additionally, the pharmaceutical compositions may be designed to delay release of the active compound over a given period of time, or to carefully control the amount of active compound released at a given time during the course of therapy.

In another embodiment, the invention methods employ pharmaceutical compositions including at least one compound of Structure 1 in a pharmaceutically acceptable carrier therefor. The phrase “pharmaceutically acceptable” means the carrier, diluent or excipient must be compatible with the other ingredients of the composition and not deleterious to the recipient thereof.

As used herein, the term “therapeutically effective amount” means the amount of the pharmaceutical composition that will elicit the biological or medical response of a mammal in need thereof that is being sought by the researcher, veterinarian, medical doctor or other clinician. In some embodiments, the mammal is human.

Pharmaceutical compositions of the present invention can be used in the form of a solid, a solution, an emulsion, a dispersion, a micelle, a liposome, and the like, wherein the resulting composition contains at least one compound of the present invention, as an active ingredient, in admixture with an organic or inorganic carrier or excipient suitable for enteral or parenteral applications. The compounds described may be combined, for example, with the usual non-toxic, pharmaceutically acceptable carriers for tablets, pellets, capsules, suppositories, solutions, emulsions, suspensions, and any other form suitable for use. The carriers which can be used include glucose, lactose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening and coloring agents and perfumes may be used. The compounds described herein are included in the pharmaceutical composition in an amount sufficient to produce the desired effect upon the process or disease condition.

Pharmaceutical compositions of the present invention may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of a sweetening agent such as sucrose, lactose, or saccharin, flavoring agents such as peppermint, oil of wintergreen or cherry, coloring agents and preserving agents in order to provide pharmaceutically elegant and palatable preparations. Tablets containing the compounds described herein in admixture with non-toxic pharmaceutically acceptable excipients may also be manufactured by known methods. The excipients used may be, for example, (1) inert diluents such as calcium carbonate, lactose, calcium phosphate or sodium phosphate; (2) granulating and disintegrating agents such as corn starch, potato starch or alginic acid; (3) binding agents such as gum tragacanth, corn starch, gelatin or acacia, and (4) lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

In some cases, formulations for oral use may be in the form of hard gelatin capsules wherein the invention compounds are mixed with an inert solid diluent, for example, calcium carbonate, calcium phosphate or kaolin. They may also be in the form of soft gelatin capsules wherein the invention compounds are mixed with water or an oil medium, for example, peanut oil, liquid paraffin, or olive oil.

The pharmaceutical compositions may be in the form of a sterile injectable suspension. This suspension may be formulated according to known methods using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Sterile, fixed oils are conventionally employed as a solvent or suspending medium. For this purpose any bland fixed oil may be employed including synthetic mono- or diglycerides, fatty acids (including oleic acid), naturally occurring vegetable oils like sesame oil, coconut oil, peanut oil, cottonseed oil, etc., or synthetic fatty vehicles like ethyl oleate or the like. Buffers, preservatives, antioxidants, and the like can be incorporated as required.

The pharmaceutical compositions described herein may also be administered in the form of suppositories for rectal administration of the drug. These compositions may be prepared by mixing the compounds described herein with a suitable non-irritating excipient, such as cocoa butter, synthetic glyceride esters of polyethylene glycols, which are solid at ordinary temperatures, but liquefy and/or dissolve in the rectal cavity to release the drug.

Since individual subjects may present a wide variation in severity of symptoms and each drug has its unique therapeutic characteristics, the precise mode of administration and dosage employed for each mammal is left to the discretion of the practitioner.

The following examples are intended only to illustrate the invention and should in no way be construed as limiting the invention.

### EXAMPLE

### General Synthesis of Amine Precursors

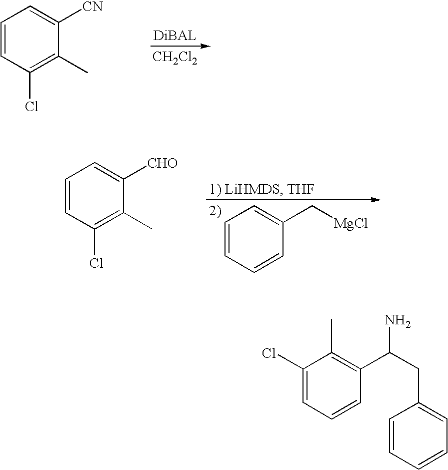

### A. 3-Chloro-2-methylbenzaldehyde

To 3-chloro-2-methylbenzonitrile (5 g, 33 mmol) in dichloromethane (150 mL) at −78° C. was added DiBAL (1M in dichloromethane, 41 mL). The reaction mixture was stirred at −78° C. for 2 h then quenched with methanol. The mixture was warmed to 0° C and HCl (10%) was added. The ice-water bath was removed and the mixture was stirred at room temperature for 10 min. The two phases were separated and aqueous phase was extracted with dichloromethane. Combined dichloromethane was washed with brine, dried over sodium sulfate and concentrated. Column chromatography (5% ethyl acetate/hexane) gave 3-chloro-2-methylbenzaldehyde (3.5 g, 69%). ¹H NMR (300 MHz, CDCl₃) δ 2.64 (s, 3H), 7.21-7.26 (m, 1H), 7.50-7.53(m, 1H), 7.63-7.66 (m, 1H), 10.20 (s, 1H)

### B. 1-(3-Chloro-2-methylphenyl)-2-phenylethanamine

To 3-chloro-2-methylbenzaldehyde (2.85 g, 18.5 mmol) in THF (5 mL) at 0° C. was added lithium bis(trimethylsilyl)amide (1M in THF, 22.2 mL). The ice-water bath was removed and the reaction mixture was stirred from 0° C. to room temperature for 2 h. The reaction mixture was then cooled back to 0° C. and benzylmagnesium chloride (1M in THF, 22.2 mL) was added. The reaction mixture was stirred from 0° C. to room temperature for 1 h then quenched with NH4Cl (Sat.), extracted with ethyl acetate. Combined ethyl acetate was washed with brine, dried over sodium sulfate and concentrated. HCl (1.25M in methanol) was added until a pH of 2. Methanol was removed to give yellow solid. To the solid was added dichloromethane. The suspension was filtered and washed with dichloromethane to yield white solid. The white solid was dissolved in methanol, basified with NaOH (1N) and extracted with ethyl acetate. Combined ethyl acetate was washed with brine, dried over sodium sulfate and concentrated to produce 1-(3-chloro-2-methylphenyl)-2-phenylethanamine (22.73 g, 60%) as a light yellow oil. ¹H NMR (300 MHz, CDCl₃) δ 2.37 (s, 3H), 2.67-2.75 (m, 1H), 2.95-3.01 (m, 1H), 4.46-4.50 (m, 1H), 7.17-7.19 (m, 3H), 7.24-7.33(m, 4H), 7.46-7.49 (m, 1H).

### General Synthesis of the Aminoimidazolines, Aminooxazolines and Aminothiazolines

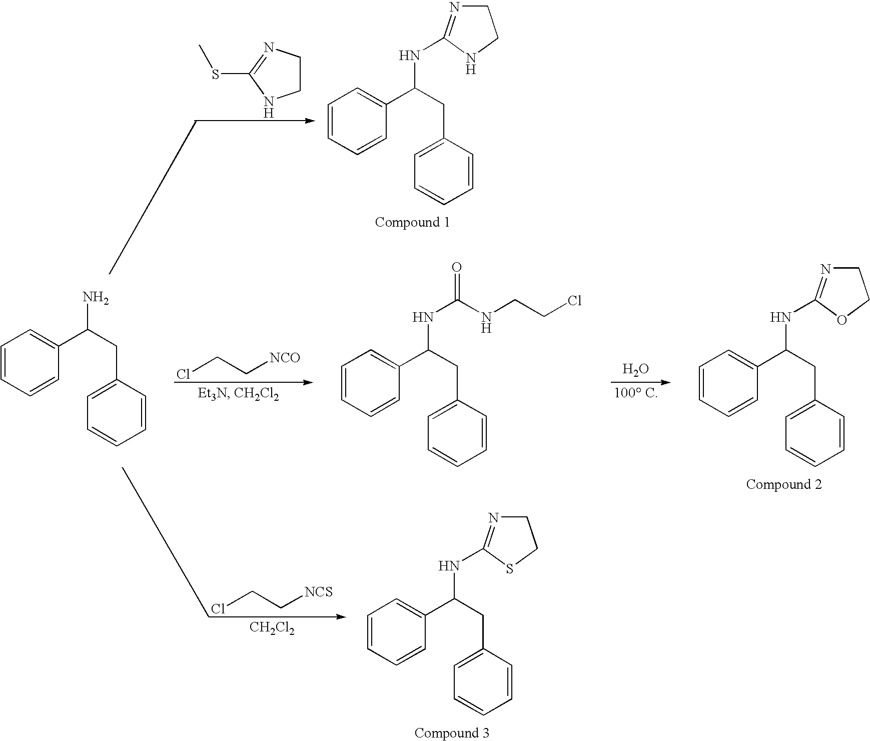

### Synthesis of N-(1,2-diphenylethyl)-4,5-dihydro-1H-imidazol-2-amine, Compound 1

A solution of 1,2-diphenylethylamine (7.0 g, 35.5 mmol) and 2-methylthio-2-imidazoline hydroiodide (5.0 g, 39.1 mmol) in isopropanol (50 mL) was heated to reflux for 16 h. The reaction mixture was concentrated and recrystalized from ether to afford N-(1,2-diphenylethyl)-4,5-dihydro-1H-imidazol-2-amine, Compound 1. ¹H NMR (300 MHz, DMSO) δ 2.90-3.14 (m, 2H), 3.36 (s, 4H), 4.78 (dd, J=8.21, 6.45 Hz, 1H), 6.87-7.55 (m, 10H).

### Synthesis of N-(1,2-diphenylethyl)-4,5-dihydrooxazol-2-amine, Compound 2

### A. 1-(2-Chloroethyl)-3 (1,2-diphenylethyl)urea

To 1,2-diphenyl-ethylamine (818 mg, 4.14 mmol) in dichloromethane (5 mL) was added 2-chloroethyl isocyanate (0.53 mL, 6.21 mmol) and triethylamine (0.86 mL). The mixture was stirred at room temperature for 1.5 h. Dichloromethane was removed and column chromatography (2-3% MeOH/CH₂Cl₂) gave 1-(2-chloroethyl)-3(1,2-diphenylethyl)urea (905 mg, 72%) as white solid. ^{H NMR (}300 MHz, CDCl₃) δ 2.98-3.00 (m, 2H), 3.30-3.41 (m, 4H), 4.89-4.96 (m, 1H), 7.00-7.03 (m, 2H), 7.17-7.30 (m, 8H).

### B. N-(1,2-diphenylethyl)-4,5-dihydrooxazol-2-amine, Compound 2

A solution of 1-(2-chloroethyl)-3(1,2-diphenylethyl)urea (543 mg, 1.8 mmol) in water (5 mL) was heated at 100° C. for 1.5 h. The reaction mixture was cooled to room temperature and sodium carbonate (sat.) was added until pH>8. The mixture was extracted with ethyl acetate. Combined ethyl acetate was washed with brine, dried over sodium sulfate and concentrated. Column chromatography (5% 7N NH₃ in MeOH/CH₂Cl₂) gave N-(1,2-diphenylethyl)-4,5-dihydrooxazol-2-amine, Compound 2, (381 mg, 80%) as white solid. ¹H NMR (300 MHz, CDCl₃) δ 3.03-3.06 (m, 2H), 3.60-3.66 (m, 2H), 4.07-4.13 (m, 2H), 4.86-4.90 (m, 1H), 7.03-7.06 (m, 2H), 7.15-7.25 (m, 8H).

### N-(1,2-diphenylethyl)-4,5-dihydrothiazol-2-amine, Compound 3

To 1,2-diphenylethylamine (818 mg, 4.14 mmol) in dichloromethane (5 mL) was added 2-chloroethyl isothiocyanate (0.097 mL, 0.99 mmol). The mixture was stirred at room temperature for 1 h. Dicholormethane was removed and column chromatography (6% MeOH/CH₂Cl₂) gave N-(1,2-diphenylethyl)-4,5-dihydrothiazol-2-amine, Compound 3, (330 mg, 29%) as a white solid. ¹H NMR (300 MHz, CDCl₃) δ 3.10-3.16 (m, 1H), 3.24-3.87 (m, 3H), 3.81-3.87 (m, 2H), 4.51-4.56 (m, 1H), 7.18-7.34 (m, 10H).

### N-(1-(3-Chlorophenyl)-2-m-tolylethyl)-4,5-dihydrooxazol-2-amine, Compound 4

¹H NMR (300 MHz, CDCl₃) δ 2.29(s, 3H), 2.94-3.06(m, 2H), 3.65-3.71 (m, 2H), 4.16-4.22 (m, 2H), 4.83-4.88(m, 1H), 6.83-6.87 (m, 2H), 7.01-7.04(m, 1H), 7.09-7.16 (m, 2H), 7.20-7.26 (m, 3H).

### N-(1-(3-Chlorophenyl)-2-m-tolylethyl)-4,5-dihydrothiazol-2-amine, Compound 5

¹H NMR (300 MHz, CDCl₃) δ 2.28(s, 3H), 2.98-3.12(m, 2H), 3.21-3.26 (m, 2H), 3.81-3.86 (m, 2H), 4.70-4.74(m, 1H), 6.87-6.90 (m, 2H), 7.00-7.03(m, 1H), 7.11-7.18 (m, 3H), 7.20-7.26 (m, 2H).

### N-(1-(3-Chlorophenyl)-2-phenylethyl)-4,5-dihydrooxazol-2-amine, Compound 6

¹H NMR (300 MHz, CDCl₃) δ 3.03-3.06 (m, 2H), 3.66-3.72 (m, 2H), 4.17-4.23 (m, 2H), 4.86-4.90 (m, 1H), 7.05-7.12 (m, 3H), 7.1227.28 (m, 6H).

### N-(1-(3-Chlorophenyl)-2-phenylethyl)-4,5-dihydrothiazol-2-amine, Compound 7

¹H NMR (300 MHz, CDCl₃) δ 3.06-3.08 (m, 2H), 3.23-3.28 (m, 2H), 3.85-3.90 (m, 2H), 4.87-4.92 (m, 1H), 7.03-7.10(m, 3H), 7.21-7.28 (m, 6H).

### Synthesis of N-(1-(3-Chlorophenyl)-2-phenylethyl)-4,5-dihydro-1H-imidazol-2-amine, Compound 8

To a solution of 1-(3-chlorophenyl)-2-phenylethylamine (400 mg, 1.73 mmol) in acetonitrile (5 mL) was added 4,5-dihydro-1H-imidazole-2-sulfonic acid (260 mg, 1.73 mmol) and triethylamine (0.24 mL). The mixture was heated at 70° C. for 45 min. The reaction mixture was cooled to room temperature. The white solid was filtered off and washed with acetonitrile to give N-(1-(3-chlorophenyl)-2-phenylethyl)-4,5-dihydro-1H-imidazol-2-amine, Compound 8 (272 mg, 53%). ¹H NMR (300 MHz, CD₃OD) δ 3.00-3.07 (m, 1H), 3.13-3.21 (m, 1H), 3.52 (s, 4H), 4.62-4.67 (m, 1H), 7.18-7.36 (m, 9H).

The following compounds were synthesized by one of the general methods described above.

### N-(1-(3-Chlorophenyl)-2-m-tolylethyl)-4,5-dihydro-1H-imidazol-2-amine, Compound 9

¹H NMR (300 MHz, CD₃OD) δ 2.26(s, 3H), 2.94-2.98 (m, 2H), 3.39 (s, 4H), 4.69-4.74 (m, 1H), 6.92-6.99(m, 3H), 7.07-7.12(m, 1H), 7.18-7.25 (m, 3H), 7.30(s, 1H).

### N-(1-(3-Chlorophenyl)-2-p-tolylethyl)-4,5-dihydrothiazol-2-amine, Compound 10

¹H NMR (300 MHz, CDCl₃) δ 2.30(s, 3H), 3.00-3.03 (m, 2H), 3.22-3.26 (m, 2H), 3.85-3.90 (m, 2H), 4.86-4.90 (m, 1H), 6.91-6.93(m, 2H), 7.04-7.09(m, 3H), 7.20-7.26 (m, 3H).

### N-(1-(3-Chlorophenyl)-2-p-tolylethyl)-4,5-dihydro-1H-imidazol-2-amine, Compound 11

¹H NMR (300 MHz, CD₃OD) δ 2.26(s, 3H), 2.95-3.01(m, 1H), 3.08-3.16 (m, 1H), 3.52 (s, 4H), 4.57-4.62 (m, 1H), 7.03-7.10(m, 4H), 7.24-7.34 (m, 4H).

### N-(1-(3-Chlorophenyl)-2-o-tolylethyl)-4,5-dihydro-1H-imidazol-2-amine, Compound 12

¹H NMR (300 MHz, CD₃OD) δ 2.26(s, 3H), 3.10-3.13 (m, 2H), 3.58 (s, 4H), 4.70-4.75 (m, 1H), 7.07-7.15(m, 4H), 7.21-7.24(m, 1H), 7.29-7.34 (m, 3H).

### N-(1-(2,3-Dimethylphenyl)-2-phenylethyl)-4,5-dihydro-1H-imidazol-2-amine, Compound 13

¹H NMR (300 MHz, CD₃OD) 6 2.20(s, 3H), 2.27(s, 3H), 2.99-3.03 (m, 2H), 3.49 (s, 4H), 4.86-4.92 (m, 1H), 7.09-7.11(m, 2H), 7.20-7.33 (m, 6H).

### N-(1-(3-Chlorophenyl)-2-p-tolylethyl)-4,5-dihydrooxazol-2-amine, Compound 14

¹H NMR (300 MHz, CDCl₃) δ 2.30(s, 3H), 2.91-3.04(m, 2H), 3.65-3.71 (m, 2H), 4.11-4.20 (m, 2H), 4.84-4.89(m, 1H), 6.91-6.93 (m, 2H), 7.04-7.10(m, 3H), 7.20-7.26 (m, 3H).

### N-(1-(3-Chlorophenyl)-2-o-tolylethyl)-4,5-dihydrothiazol-2-amine, Compound 15

¹H NMR (300 MHz, CDCl₃) δ 2.20(s, 3H), 3.02-3.06 (m, 2H), 3.20-3.25 (m, 2H), 3.81-3.86 (m, 2H), 4.78-4.83 (m, 1H), 6.97-6.99(m, 1H), 7.04-7.12(m, 4H), 7.19-7.21 (m, 3H).

### N-(1-(2,3-Dimethylphenyl)-2-phenylethyl)-4,5-dihydrothiazol-2-amine, Compound 16

¹H NMR (300 MHz, CDCl₃) δ 2.15(s, 3H), 2.26(s, 3H), 3.05-3.08 (m, 2H), 3.19-3.24 (m, 2H), 3.84-3.89 (m, 2H), 5.06-5.11 (m, 1H), 7.06-7.13(m, 4H), 7.18-7.27 (m, 4H).

### N-(1-(3-Chlorophenyl)-2-o-tolylethyl)-4,5-dihydrooxzol-2-amine, Compound 17

¹H NMR (300 MHz, CDCl₃) δ 2.21(s, 3H), 2.99-3.03(m, 2H), 3.64-3.70 (m, 2H), 4.14-4.20 (m, 2H), 4.83-4.88(m, 1H), 6.98-7.01 (m, 1H), 7.07-7.14(m, 4H), 7.20-7.26 (m, 3H).

### N-(1-(3-Chloro-2-fluorophenyl)-2-phenylethyl)-4,5-dihydro-1H-imidazol-2-amine, Compound 18

¹H NMR (300 MHz, CD₃OD) δ 3.04-3.08 (m, 2H), 3.44 (s, 4H), 5.02-5.06 (m, 1H), 7.09-7.40(m, 8H).

### N-(1-(3-Chloro-2-methylphenyl)-2-phenylethyl)-4,5-dihydro-1H-imidazol-2-amine, Compound 19

¹H NMR (300 MHz, CD₃OD) δ 2.32 (s, 3H), 3.02-3.05 (m, 2H), 3.50 (s, 4H), 4.85-4.89 (m, 1H), 7.17-7.32 (m, 7H), 7.52-7.55 (m, 1H).

### N-(1-(2,3-Dimethylphenyl)-2-phenylethyl)-4,5-dihydrooxazol-2-amine, Compound 20

¹H NMR (300 MHz, CDCl₃) δ 2.18(s, 3H), 2.25(s, 3H), 2.97-3.01(m, 2H), 3.63-3.68 (m, 2H), 4.07-4.13 (m, 2H), 5.16-5.21(m, 1H), 7.03-7.09(m, 5H), 7.12-7.23 (m, 3H).

### N-(1-(3-Chloro-2-fluorophenyl)-2-phenylethyl)-4,5-dihydrooxazol-2-amine, Compound 21

¹H NMR (300 MHz, CDCl₃) δ 3.04-3.13(m, 2H), 3.64-3.70 (m, 2H), 4.13-4.19 (m, 2H), 5.12-5.17(m, 1H), 6.94-7.08(m, 4H), 7.19-7.30 (m, 4H).

### N-(1-(3-Chloro-2-fluorophenyl)-2-phenylethyl)-4,5-dihydrothiazol-2-amine, Compound 22

¹H NMR (300 MHz, CDCl₃) δ 3.10-3.12 (m, 2H), 3.21-3.26 (m, 2H), 3.82-3.87 (m, 2H), 5.07-5.12 (m, 1H), 6.99-7.04(m, 1H), 7.09-7.16(m, 3H), 7.20-7.31 (m, 4H).

### N-(1-(2,3-Dichlorophenyl)-2-phenylethyl)-4,5-dihydro-1H-imidazol-2-amine, Compound 23

¹H NMR (300 MHz, CD₃OD) δ 2.94-3.02(m, 1H), 3.15-3.21(m, 1H), 3.49 (s, 4H), 5.02-5.06 (m, 1H), 7.19-7.41(m, 6H), 7.47-7.51(m, 1H), 7.64-7.68(m, 1H).

### N-(1-(2,3-Dichlorophenyl)-2-(4-fluorophenyl)ethyl)-4,5-dihydro-1H-imidazol-2-amine, Compound 24

¹H NMR (300 MHz, CD₃OD) δ 2.91-2.96(m, 1H), 3.21-3.24(m, 1H), 3.53 (s, 4H), 5.06-5.09 (m, 1H), 7.02-7.06(m, 2H), 7.31-7.38(m, 3H), 7.43-7.44(m, 1H), 7.52-7.54(m, 1H).

### N-(2-(2-Bromophenyl)-1-(2,3-dichlorophenyl)ethyl)-4,5-dihydro-1H-imidazol-2-amine, Compound 25

¹H NMR (300 MHz, CD₃OD) δ 3.21-3.25(m, 2H), 3.43 (s, 4H), 5.28-5.32 (m, 1H), 7.12-7.14(m, 1H), 7.19-7.23(m, 2H), 7.29-7.32(m, 1H), 7.42-7.47(m, 2H), 7.53-7.56(m, 1H).

### N-(1-(2,3-Dichlorophenyl)-2-(3-methoxyphenyl)ethyl)-4,5-dihydro-1H-imidazol-2-amine, Compound 26

¹H NMR (300 MHz, CD₃OD) δ 2.91-2.96(m, 1H), 3.25-3.28(m, 1H), 3.57 (s, 4H), 3.78(s, 3H), 5.10-5.13 (m, 1H), 6.83-6.86(m, 2H), 6.89-6.91(m, 1H), 7.23-7.26(m, 1H), 7.37-7.43(m, 2H), 7.55-7.57(m, 1H).

### N-(1-(2,3-Dichlorophenyl)-2-(3-fluoro2-methylphenyl)ethyl)-4,5-dihydro-1H-imidazol-2-amine, Compound 27

¹H NMR (300 MHz, CD₃OD) δ 2.28(s, 3H), 3.11-3.16(m, 1H), 3.29-3.33(m, 1H), 3.57 (s, 4H), 5.14-5.17 (m, 1H), 6.95-7.01(m, 2H), 7.12-7.16(m, 1H), 7.38-7.41(m, 1H), 7.46-7.47(m, 1H), 7.56-7.58(m, 1H).

### N-(1-(2,3-Dichlorophenyl)-2-(2,5-dichlorophenyl)ethyl)-4,5-dihydro-1H-imidazol-2-amine, Compound 28

¹H NMR (300 MHz, CD₃OD) δ 3.09-3.17(m, 1H), 3.24-3.34(m, 1H), 3.45 (s, 4H), 5.25-5.30 (m, 1H), 7.21-7.25(m, 1H), 7.29-7.36(m, 3H), 7.44-7.49(m, 2H).

### N-(2-(2-Chloro-6-fluorophenyl)-1-(2,3-dichlorophenyl)ethyl)-4,5-dihydro-1H-imidazol-2-amine, Compound 29

¹H NMR (300 MHz, CD₃OD) δ 3.25-3.29(m, 2H), 3.38 (s, 4H), 5.29-5.34 (m, 1H), 6.94-7.00(m, 1H), 7.17-7.28(m, 3H), 7.40-7.47(m, 2H).

### N-(1-(2,3-Dichlorophenyl)-2-(3,5-dichlorophenyl)ethyl)-4,5-dihydro-1H-imidazol-2-amine, Compound 30

¹H NMR (300 MHz, CD₃OD) δ 2.85-2.93(m, 1H), 3.13-3.20(m, 1H), 3.46 (s, 4H), 5.11-5.14 (m, 1H), 7.26-7.27(m, 2H), 7.31-7.36(m, 2H), 7.48-7.51(m, 2H).

### N-(1-(2,3-Dichlorophenyl)-2-(2,4-dichlorophenyl)ethyl)-4,5-dihydro-1H-imidazol-2-amine, Compound 31

¹H NMR (300 MHz, CD₃OD) δ 3.09-3.16(m, 1H), 3.21-3.28(m, 1H), 3.42 (s, 4H), 5.24-5.29 (m, 1H), 7.22-7.23(m, 2H), 7.27-7.33(m, 1H), 7.41-7.48(m, 3H).

### N-(1-(2,3-Dichlorophenyl)-2-(3,4-dichlorophenyl)ethyl)-4,5-dihydro-1H-imidazol-2-amine Compound 32

¹H NMR (300 MHz, CD₃OD) δ 2.80-2.87(m, 1H), 3.10-3.16(m, 1H), 3.40 (s, 4H), 5.10-5.14 (m, 1H), 7.18-7.21(m, 1H), 7.28-7.33(m, 2H), 7.41-7.48(m, 3H).

### 3(2-(2,3-Dichlorophenyl)-2-(4,5-dihydro-1H-imidazol-2-ylamino)ethyl)phenol Compound 33

¹H NMR (300 MHz, CD₃OD) δ 2.75-2.83(m, 1H), 3.12-3.18(m, 1H), 3.51 (s, 4H), 5.05-5.09 (m, 1H), 6.62-6.74(m, 3H), 7.05-7.11(m, 1H), 7.31-7.44(m, 2H), 7.50-7.53(m, 1H).

### N-(1-(3-Chlorophenyl)-2-(2-methoxyphenyl)ethyl)-4,5-dihydro-1H-imidazol-2-amine Compound 34

¹H NMR (300 MHz, CD₃OD) δ 2.98-3.14(m, 2H), 3.53(s, 4H), 3.84 (s, 3H), 4.77-4.82 (m, 1H), 6.80-6.85(m, 1H), 6.93-6.96(m, 1H), 7.03-7.06(m, 1H), 7.19-7.34(m, 5H).

### N-(1-(3-Chlorophenyl)-2-(2-methoxyphenyl)ethyl)-4,5-dihydrooxazol-2-amine Compound 35

¹H NMR (300 MHz, CD₃COCD₃) δ 3.02-3.04(m, 2H), 3.45-3.51 (m, 2H), 3.85(s, 3H), 4.03-4.08 (m, 2H), 4.87-4.92(m, 1H), 6.80-6.85(m, 1H), 6.94-6.96(m, 1H), 7.12-7.23(m, 3H), 7.28-7.30 (m, 2H), 7.39-7.40(m, 1H).

### N-(2-(2-Bromophenyl)-1-(3-chlor-phenyl)ethyl)-4,5-dihydro-1H-imidazol-2-amine Compound 36

¹H NMR (300 MHz, CD₃OD) δ 3.14-3.16(m, 2H), 3.40 (s, 4H), 4.80-4.85 (m, 1H), 7.06-7.11(m, 1H), 7.19-7.27(m, 4H), 7.36(m, 1H), 7.51-7.54(m, 1H).

### N-(2-(2-Bromophenyl)-1-(3-chlor-phenyl)ethyl)-4,5-dihydrooxazol-2-amine Compound 37

¹H NMR (300 MHz, CD₃OD) δ 3.03-3.15(m, 2H), 3.50-3.56 (m, 2H), 4.15-4.21 (m, 2H), 4.85-4.90(m, 1H), 7.08-7.27(m, 6H), 7.33-7.35(m, 1H), 7.53-7.56 (m, 1H).

While this invention has been described with respect to these specific examples, it is understood that other modifications and variations are possible without departing from the spirit of the invention.

What is claimed is:

1. A method of treating pain comprising administering to a mammal in need thereof a pharmaceutical composition containing a therapeutically effective dose of at least one compound having the structure: 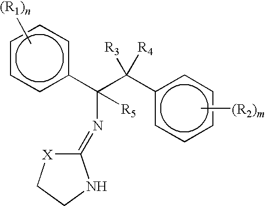
wherein:
X is O, S, or NH;
n and m are each independently 1 to 5;
each R₁ and R₂ is independently H, alkyl, cycloalkyl, aryl, alkenyl, alkynyl, halide, hydroxy, alkoxy, trifluoromethyl, —N(R₆)₂, —CN, —CO₂R₆, or —CH₂OH; and
R₃, R₄, R₅, and R₆ are each independently H or lower alkyl;
or any combination thereof, or pharmaceutically acceptable salts, hydrates, solvates, crystal forms, isomers, tautomers, enantiomers, and diastereomers thereof.

2. The method of claim 1 wherein each R₁ and R₂ is independently H, lower alkyl, fluoro, chloro, bromo, trifluoromethyl, hydroxy, or methoxy.

3. The method of claim 1 wherein X is S.

4. The method of claim 3 wherein each R₁ and R₂ is independently H, lower alkyl, fluoro, chloro, bromo, trifluoromethyl, hydroxy, or methoxy.

5. The method of claim 4 wherein the compound has the structure

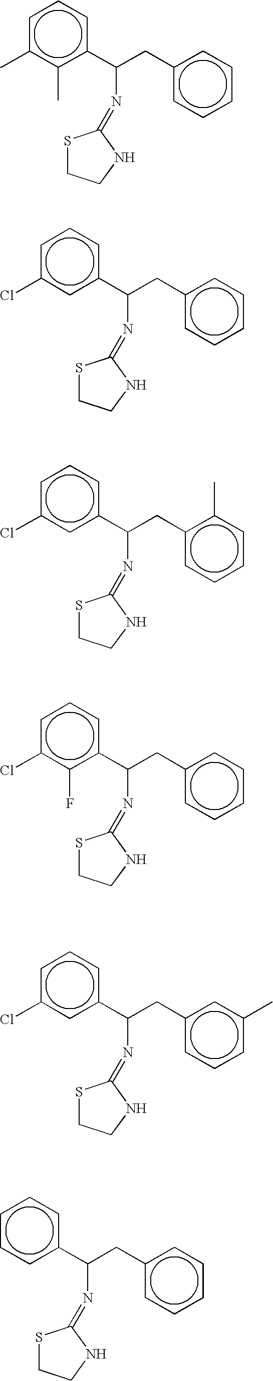

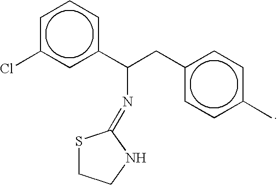

6. The method of claim 1 wherein X is NH.

7. The method of claim 6 wherein each R₁ and R₂ is independently H, lower alkyl, fluoro, chloro, bromo, trifluoromethyl, hydroxy, or methoxy.

8. The method of claim 7 wherein the compound has the structure

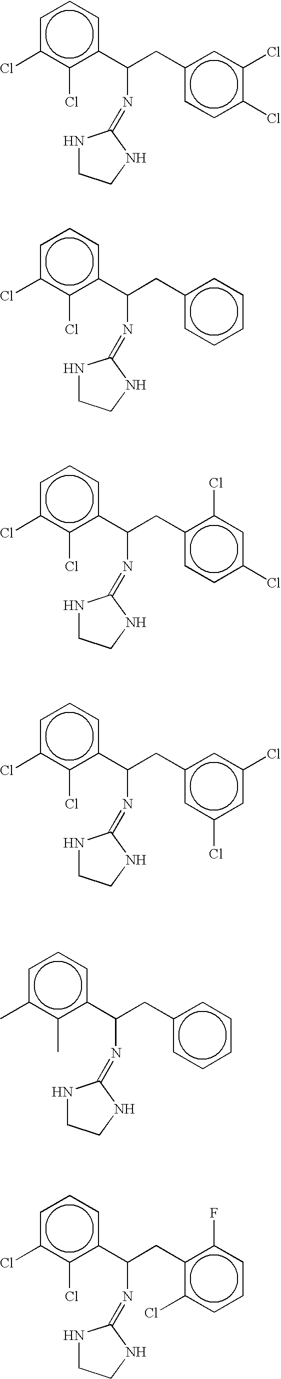

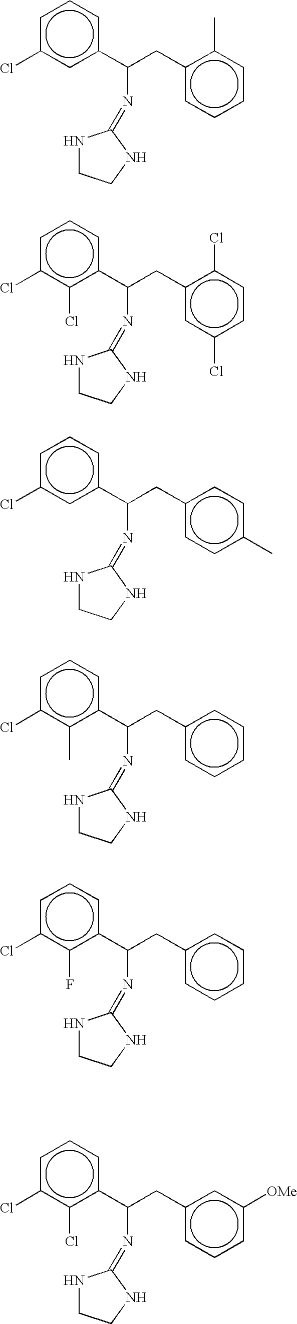

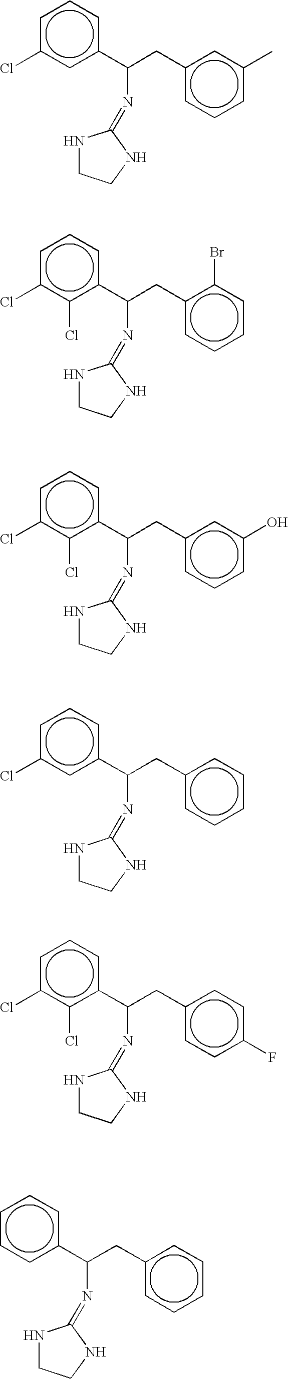

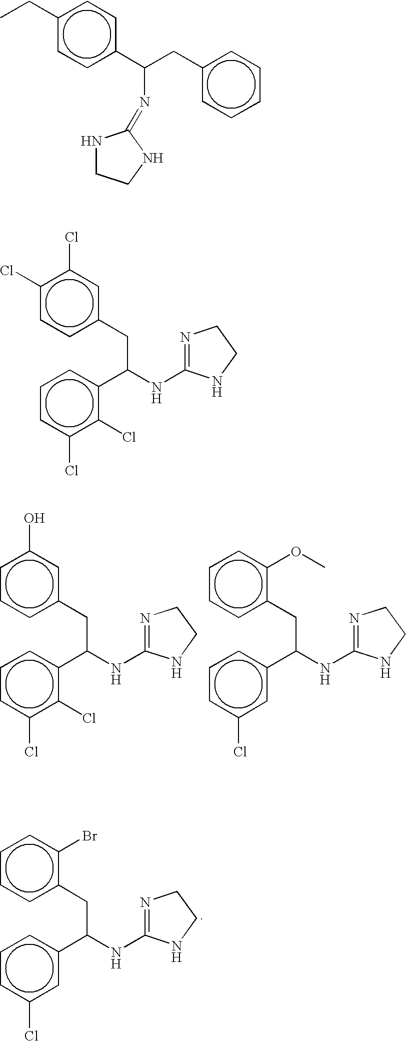

9. The method of claim 1 wherein X is O.

10. The method of claim 9 wherein each R₁ and R₂ is independently H, lower alkyl, fluoro, chloro, bromo, trifluoromethyl, hydroxy, or methoxy.

11. The method of claim 9 wherein the compound has the structure

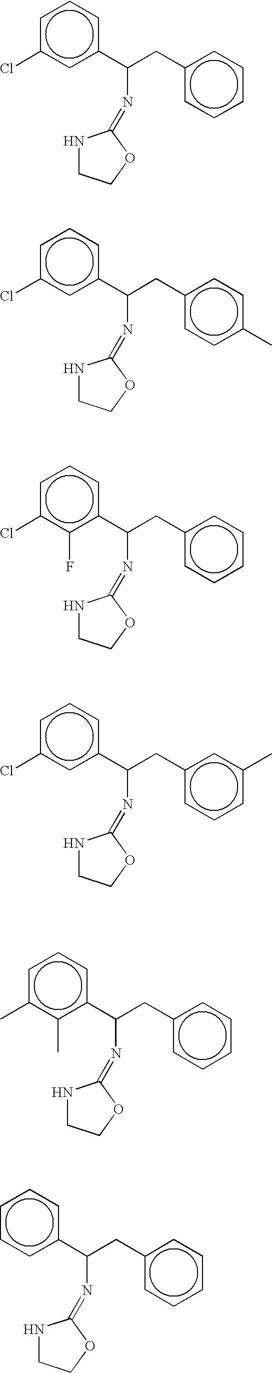

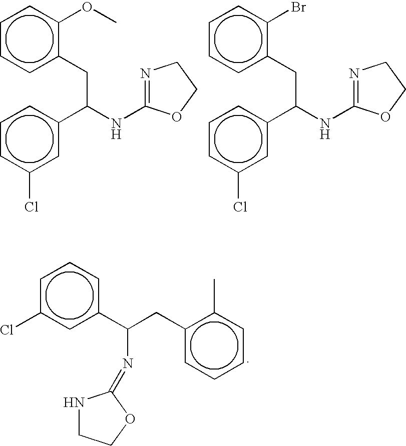

12. The method of claim 1, wherein the pharmaceutical composition is administered to the mammal to treat neuropathic pain, chronic pain, or visceral pain.

13. The method of claim 1, wherein the pharmaceutical composition is administered to the mammal to treat allodynia.
